(19) United States
(12) Patent Application Publication
(10) Pub. No.: US 2009/0165804 A1
(43) Pub. Date: Jul. 2, 2009

(54) Ear pros

(76) Inventor: Ruth Crossley Maxwell, Jefferson, LA (US)

Correspondence Address: Ruth C. Maxwell, 916 Claiborne Dr., Jefferson, LA, 70121, US

(21) Appl. No.: 12/005,934

(22) Filed: Dec. 31, 2007

(51) Int. Cl. A61F 11/00

(52) U.S. Cl. 128/857

(57) ABSTRACT

The “EAR PROS” are protectors for the ears, made of a polyester or light weight Material. It has an elastic band (draw string) for securing and sizing this invention to the Ears. “EAR PROS” come as a pair, interchangeable for the right or left ear. Both ears should be covered to protect them from the hair drying Utensil's hot air.

It may used by male or female.

### BACKGROUND OF THE INVENTION

Being subjected to the hot air from a hair drying utensil, physical or emotional scars are possible; this invention will endeavor to assist in that area.

When a hair drying utensil is being used for any prolonged period of time on the head, the ears remain a prime target, for discomfort or even pain.

The hot air from a hair drying utensil may result with burns on the ear(s) and/or severe discomfort.

### DETAILED DESCRIPTION OF THE INVENTION

A beauty hair salon product for ear protection:
1. Made of synthetic fabric or terry cloth fabric
2. Dimensions 4×5 inches
3. Fabric cut in the shape of a horse shoe (except; no hollow center)
4. 19 inch elastic band

### BRIEF SUMMARY OF THE INVENTION

Through this invention, it will allow each ear to be covered with a comfortable fabric. The invention will engulf or cup the ears, therefore protecting them from the hot air of a hair drying utensil, e.g. hair dryer.

With this new beauty salon product, all humanity will benefit, whenever obliged using a hair drying utensil.

Also, this invention, named (EAR PROS) will be a great asset to the Cosmopolitan Industry.

1. What I claim as my invention, is to be used as a pair, one identical, for each ear, Adjustable to fit. This invention cover the ears for their protection while drying a Person's hair:
The invention is called “EAR PROS”, which stands for ear protectors.
This patent should protect my invention for 20 (twenty) years; excluding others from Making, using, offering for sale or selling the invention throughout the United States or Importing the invention into the United States and its territory from the date which this patent application is filed.
